(19) United States
(12) Patent Application Publication
(10) Pub. No.: US 2007/0277842 A1
(43) Pub. Date: Dec. 6, 2007

(54) Hair marker instrument

(76) Inventors: April K. Bay-Hinitz, Reno, NV (US); David Hinitz, Reno, NV (US)

Correspondence Address: SUNG I. OH, PROFESSIONAL LAW CORPORATION, 710 QUAIL VALLEY LANE, WEST COVINA, CA, 91791, US

(21) Appl. No.: 11/444,230

(22) Filed: May 30, 2006

(51) Int. Cl. A61K 8/18

(52) U.S. Cl. 132/208

(57) ABSTRACT

A pencil-like stick has hair color therein and is used in the manner of a magic marker to apply to a color to a user's hair. The stick includes a tip having two arms that are closed by a lever on the stick to trap strands of hair therebetween. When hair is trapped between the two arms, hair color from the stick is wicked from a reservoir in the stick to the hair.

### TECHNICAL FIELD OF THE INVENTION

The present invention relates to the general art of hair accessories, and to the particular field of accessories used to color hair.

### BACKGROUND OF THE INVENTION

It has been estimated that during any given year, about 43.million visits are made by clients to beauty shops in the United States. A substantial number of these visits involve changing the color of the client's hair. Thus, the coloring of hair is an important volume business, and it is estimated that the hair dye industry grosses 280 million dollars a year.

While it is relatively easy to change the color of the hair as a whole, by dyeing to make it darker, or by bleaching to lighten it the result of only dyeing or only bleaching may be disappointing. The hair so treated tends to have an uninteresting uniform color. It is customary to follow dyeing or bleaching operation with another step, in which selected strands of hair are further treated so that their color will change and so that these selected strands will stand out against the background provided by the remainder of the hair. Similarly, selected strands of hair of a person's natural hair can be treated.

Treatment of selected strands of hair to change the color is called streaking if the selected strands are lightened, or reverse streaking if they are darkened. Other terms such as frosting and tipping are related and will be discussed below. Streaking can produce an appearance similar to that of highlights glistening on the hair, and is much desired. Reverse streaking can give an of texture to dull hair. It can also be used when the client wishes to return to her or his own natural shade. In this case, as the roots grow out, the previously beached hair can be reverse streaked to make less apparent the difference between the different portions of hair.

The task of coloring selected strands of hair is difficult for the hair stylist, since many strands of hair all over the head must be individually treated with appropriate treating material, while guarding the general mass of hair from contact with the treating material, and it is arduous for the client because of the length of time involved.

Indeed, in the frosting cap method, the procedure is actually painful to the client. In this method the hair is first combed, then covered with a thin film of plastic and finally covered with a heavy rubber cap, which is provided with a large number of small holes. The hair stylist uses a smooth crochet hook to punch through the plastic film at each-hole in the rubber cap, snares the hair which lies underneath the hole and fishes it out with the hook. Considerable force must be used to fish out the selected strands of hair because of the adjacency of other strands, and the confinement of the plastic film and rubber cap. After the selected strands are pulled out and exposed, outside of the rubber cap, they are treated with treating material, which is kept from reaching the scalp and the remaining hair by the tight fit of the rubber cap and plastic film.

The frosting cap method is popular and has certain advantages. There is no contact of the scalp with the chemicals used, and the treated hair is well segregated from the hair which is not to be treated. However, besides the painful aspects mentioned above, there is much breakage of hair, the hair stylist has limited control of which particular strands of hair are pulled out, and the rubber cap must stay in place for the wearisome time of up to an hour.

Another method, the “Dixie Cup” method, utilizes cups. Selected strands of hair are pulled through a small hole in the bottom of the cup into the interior thereof, the selected strands are then treated and packed into the cup. The method permits greater control by the hair stylist of the choice of which particular strands of hair are to be treated than does the frosting cap method. However the size of the cups dictates that the different treated strands must be widely spaced, which is a disadvantage. This method is time consuming since each cup must be individually handled. It is difficult to manipulate short hair into the cup. The Dixie Cup method has not gained great popularity.

In the foil method of coloring hair, the hair stylist isolates a strand of hair from the remaining hair, and lays it over a piece of foil which is butted up against the scalp, adjacent the roots of the isolated strand. The isolated strand is then treated with treating material, and the foil is folded around it to act as a barrier against migration of the treating material. It is difficult for the hair stylist using this method to follow the progress of the treatment since the hair being treated is hidden. When streaking, some of the hair is sometimes over bleached.

Another method is a weaving comb method. Briefly, the method uses a weaving comb, which has gullets of two depths between its teeth. When the comb is used, it acts like the headles of a loom to separate the hair being combed into an upper and a lower flight, with a shed between, and with the upper flight flowing through the comb adjacent the spine of the comb and the lower flight flowing through the comb closer to the tips of the teeth. Treating material is placed on the spine of the comb, adjacent the teeth. When the hair is then combed with the weaving comb, some of the treating material transfers to the hair shafts of the upper flight while the hair shafts of the lower flight are not touched by the treating material because of the separation provided by the shed. This method has the advantage of applying the treating material to the hair being combed during a single stroke of the weaving comb. However, considerable time is taken to prepare a parting of hair for the weaving comb, and the comb must be reloaded with treating material for each stroke.

None of these known methods are suitable for a person who wishes to simply touch up their hair in the manner of touching up make-up or the like. That is, if the person sees a strand of hair or a small portion of hair that does not appeal to them at that moment, the person may wish to touch up that portion of the hair in a quick, easy and efficient manner. None of the above-described methods will permit this action.

While the inventor is aware of applicator devices for applying a hair treating liquid to the hair having flexible supply containers which are manually squeezed in order to force the hair treating liquid from the container into a distributing apparatus. These typical prior art devices utilize a distributing device attached to the flexible container to distribute the hair treating fluid evenly throughout a section of the hair and scalp. The most usage of such devices is to apply a hair coloring fluid in order to alter the color of the operator's hair.

As is often the case, the hair coloring fluids contain ingredients which cause damage to the scalp of the user if applied directly thereto, instead of the hair itself. The prior art devices have no means with which to prevent the hair treating fluid from coming into contact with the scalp of the user. Also, the distributing apparatus utilized by the prior art devices has a plurality of comb-like teeth, which are integrally molded with a common supply tube attached to the flexible reservoir, this integral molding requires a complex mold apparatus and, thereby, serves to increase the cost of such devices.

Also, if one of the comb teeth should be broken off, the effectiveness of the distributing apparatus is totally destroyed of a loom to separate the hair being combed into an upper and a lower flight, with a shed between, and with the upper flight flowing through the comb adjacent the spine of the comb and the lower flight flowing through the comb closer to the tips of the teeth. Treating material is placed on the spine of the comb, adjacent the teeth. When the hair is then combed with the weaving comb, some of the treating material transfers to the hair shafts of the upper flight while the hair shafts of the lower flight are not touched by the treating material because of the separation provided by the shed. This method has the advantage of applying the treating material to the hair being combed during a single stroke of the weaving comb. However, considerable time is taken to prepare a parting of hair for the weaving comb, and the comb must be reloaded with treating material for each stroke.

### SUMMARY OF THE INVENTION

The above-discussed disadvantages of the prior art are overcome by a pencil-like stick that has hair color therein and is used in the manner of a magic marker to apply color to a user's hair. The stick includes a tip having two arms that are closed by a lever on the stick to trap strands of hair therebetween. When hair is trapped between the two arms, hair color from the stick is wicked from a reservoir in the stick to the hair.

Using the stick embodying the present invention will permit selected strands of hair to be colored in an efficient manner. This will be especially useful in touching up gray hair.

Other systems, methods, features, and advantages of the invention will be, or will become, apparent to one with skill in the art upon examination of the following figures and detailed description. It is intended that all such additional systems, methods, features, and advantages be included within this description, be within the scope of the invention, and be protected by the following claims.

### BRIEF DESCRIPTION OF THE DRAWING FIGURE

The invention can be better understood with reference to the following drawings and description. The components in the figures are not necessarily to scale, emphasis instead being placed upon illustrating the principles of the invention. Moreover, in the figures, like referenced numerals designate corresponding parts throughout the different views.

FIG. 1 is an elevational view of a hair color applying stick embodying the present invention.

### DETAILED DESCRIPTION OF THE INVENTION

Referring to the figure, it can be understood that the present invention is embodied in a hair color applying stick 10 which is used to apply hair color to selected strands of a user's hair. Stick 10 comprises a body 12 which can be plastic or the like and which has a first end 14 which is a forward end when the body is in use, a second end 16 which is a rear end when the body is in use and a longitudinal axis 18 which extends between first end 14 and second end 16.

A hollow chamber 20 is defined in the body and a reservoir 22 is located in chamber 20. Liquid hair color 24 is stored in the reservoir. Hair color 24 can be any suitable color, but is especially useful in touching up gray hair. A wick element 30 has a first end 32 located in the reservoir to be in fluid contact with the hair color in the reservoir and a second end 34 which is a tip when the wick element is in use and which is located outside of chamber 20 and is spaced apart from first end 14 of the body. Hair color in the reservoir moves through wick element 30 to second end 34 of the wick element for use by stick 10.

A hair strand clamp unit 40 is mounted on first end 14 of the body adjacent to tip 34 of the wick element. Hair strand clamp unit 40 includes a first arm 42 which has a base end 44 mounted on the body adjacent to tip 34 of the wick element and spaced apart from longitudinal axis 18 of the body, and a second end 46 spaced apart from base end 44 in the direction of longitudinal axis 18 of the body.

A second arm 50 has a base end 52 mounted on the body adjacent to tip 34 of the wick element and spaced apart from the longitudinal axis of the body and a second end 54 spaced apart from base end 52 of the second arm in the direction of the longitudinal axis of the body. Second arm 50 is spaced apart from first arm 42. Tip 34 of the wick element is located between the first and second arms.

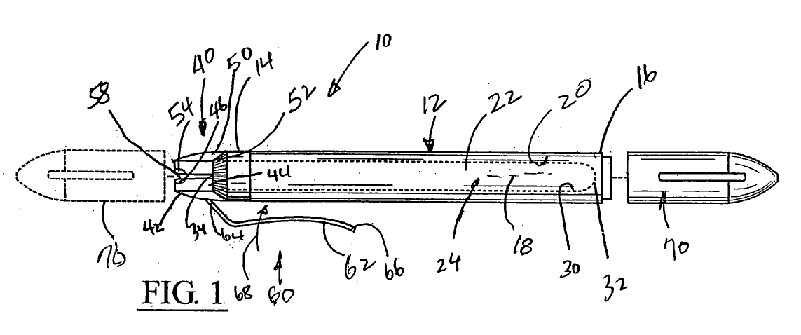

First arm 42 is flexible and is mounted to be biased into a position that has the tip of the first arm spaced apart from the tip of the second arm. First arm 42 is movable against its natural bias toward the second arm to move between a release position shown in solid lines in FIG. 1 in which the tip of first arm is spaced apart from the tip of the second arm with a gap 58 defined between the tips of the arms, and a hair clamping position in which the tip of the first arm is located closely adjacent to the tip of the second arm whereby a strand of hair will be contacted by and held in place against the tip of the wick element by the tips of the first and second arms.

An arm moving mechanism 60 is located on the body. Arm moving mechanism 60 includes a lever arm 62 having one end 64 connected to first arm 42 and a second end 66 is spaced apart from the body. Lever arm 62 is movable in direction 68 toward the body to move first arm 42 from the release position toward the hair clamping position. Once the lever arm 62 is released, the natural resiliency of first arm 42 will cause arm 42 to move back into the position shown in FIG. 1 in which the tip of the first arm is spaced apart from the tip of the second arm.

A cap element 70 is sized and adapted to cover the first and second arms of the hair strand clamp when the cap element is in position as indicated in dotted lines in FIG. 1. Clamp element 70 can be stored on the second end of the body when not in use as indicated in solid lines in FIG. 1.

Use of stick 10 can be understood from the teaching of this disclosure and will thus be only briefly discussed. A user selects a strand of hair to be colored, removes the cap and places that strand of hair between first and second arms 42 and 50 and forces lever arm 62 in direction 68 to clamp the selected strand of hair between the arms and against tip 34 of the wick element. Hair color will move through the wick element from the reservoir to tip 34 and from there will be transferred to the trapped strand of hair to color that hair. Stick 10 is drawn along the trapped hair until the desired amount of hair color has been transferred to the hair. The arms can be released once the stick has reached the distal tip of the trapped strand of hair, and re-opened to replace the stick on the trapped strand if the hair coloring process is to be repeated. These steps are repeated until the strand of hair reaches the desired color. Once the hair coloring process is completed, lever arm 62 is released and the arms will flex to move away from each other to release the strand of hair and the stick will be moved away from the strand of hair. The cap is replaced after the stick has been used to keep the wick from drying out between uses.

While various embodiments of the invention have been described, it will be apparent to those of ordinary skill in the art that many more embodiments and implementations are possible within the scope of this invention. Accordingly, the invention is not to be restricted except in light of the attached claims and their equivalents.

What is claimed is:

1. A hair color applying stick comprising:
A) a body having
(1) a first end which is a forward end when the body is in use,
(2) a second end which is a rear end when the body is in use,
(3) a longitudinal axis which extends between the first end and the second end, and
(4) a hollow chamber defined in the body;
B) a reservoir in the chamber;
C) hair color in the reservoir;
D) a wick element having a first end in the reservoir to be in fluid contact with the hair color in the reservoir and a second end which is a tip when the wick element is in use and which is located outside of the chamber and is spaced apart from the first end of the body, hair color in the reservoir moving through the wick element to the second end of the wick element;
E) a hair strand clamp unit mounted on the first end of the body adjacent to the tip of the wick element, the hair strand clamp unit including
(1) a first arm having a base end mounted on the body adjacent to the tip of the wick element and spaced apart from the longitudinal axis of the body, and a second end spaced apart from the base end in the direction of the longitudinal axis of the body,
(2) a second arm having a base end mounted on the body adjacent to the tip of the wick element and spaced apart from the longitudinal axis of the body, and a second end spaced apart from the base end of the second arm in the direction of the longitudinal axis of the body, the second arm being spaced apart from the first arm,
(3) the tip of the wick element being located between the first and second arms,
(4) the first arm being movable toward the second arm to move between a release position in which the tip of first arm is spaced apart from the tip of the second arm, a gap being defined between the tips of the arms, and a hair clamping position in which the tip of the first arm is located closely adjacent to the tip of the second arm whereby a strand of hair will be contacted by and held in place against the tip of the wick element by the tips of the first and second arms, the first arm being biased toward the release position, and
(5) an arm moving mechanism on the body, the arm moving mechanism including a lever arm having one end connected to the first arm and a second end which is spaced apart from the body, the lever arm being movable toward the body to move the first arm against the bias from the release position toward the hair clamping position; and
F) a cap element which is sized and adapted to cover the first and second arms of the hair strand clamp when the cap element is in position.

2. A hair color applying stick comprising:
A) a body having a hair color reservoir therein;
B) a wick element having a first end in the reservoir to be in fluid contact with hair color in the reservoir and a second end which is a tip when the wick element is in use and which is located outside of the body, hair color in the reservoir moving through the wick element to the second end of the wick element; and
C) a hair strand clamp mounted unit on the body adjacent to the tip of the wick element, the hair strand clamp including
(1) a first arm having a base end mounted on the body adjacent to the tip of the wick element and a second end spaced apart from the base end,
(2) a second arm having a base end mounted on the body adjacent to the tip of the wick element and a second end spaced apart from the base end of the second arm, the second arm being spaced apart from the first arm,
(3) the tip of the wick element being located between the first and second arms,
(4) the first arm being movable toward the second arm to move between a release position in which the tip of first arm is spaced apart from the tip of the second arm, a gap being defined between the tips of the arms, and a hair clamping position in which the tip of the first arm is located closely adjacent to the tip of the second arm whereby a strand of hair will be contacted by and held in place against the tip of the wick element by the tips of the first and second arms, and
(5) an arm moving mechanism on the body, the arm moving mechanism including a lever arm having one end connected to the first arm and a second end which is spaced apart from the body, the lever arm being movable toward the body to move the first arm from the release position toward the hair clamping position.

3. A method of coloring hair comprising:
A) providing a hair color applying stick comprising a body having a hair color reservoir therein; a wick element having a first end in the reservoir to be in fluid contact with hair color in the reservoir and a second end which is a tip when the wick element is in use and which is located outside of the body, hair color in the reservoir moving through the wick element to the second end of the wick element; and a hair strand clamp mounted unit on the body adjacent to the tip of the wick element, the hair strand clamp including a first arm having a base end mounted on the body adjacent to the tip of the wick element and a second end spaced apart from the base end, a second arm having a base end mounted on the body adjacent to the tip of the wick element and a second end spaced apart from the base end of the second arm, the second arm being spaced apart from the first arm, the tip of the wick element being located between the first and second arms, the first arm being movable toward the second arm to move between a release position in which the tip of first arm is spaced apart from the tip of the second arm, a gap being defined between the tips of the arms, and a hair clamping position in which the tip of the first arm is located closely adjacent to the tip of the second arm whereby a strand of hair will be contacted by and held in place against the tip of the wick element by the tips of the first and second arms, and an arm moving mechanism on the body, the arm moving mechanism including a lever arm having one end connected to the first arm and a second end which is spaced apart from the body, the lever arm being movable toward the body to move the first arm from the release position toward the hair clamping position;
B) placing a strand of hair between the tips of the first and second arms;
C) operating the lever arm to move the tip of the first arm toward the tip of the second arm and to trap the strand of hair between the tips of the first and second arms and force the trapped strand of hair against the tip of the wick element;
D) allowing hair color to move from the tip of the wick element to the trapped strand of hair; and
E) drawing the body along the trapped strand of hair to pull the wick element over the trapped strand of hair.
